(19) United States
(12) Patent Application Publication
(10) Pub. No.: US 2024/0139368 A1
(43) Pub. Date: May 2, 2024

(54) AIR PURIFYING PERFORMANCE EVALUATION DEVICE

(71) Applicant: Samsung Electronics Co., Ltd., Suwon-si (KR)

(72) Inventors: Jaeeun KIM, Suwon-si (KR); Joonseon JEONG, Suwon-si (KR); Jinkyu KANG, Suwon-si (KR); Dongwook KIM, Suwon-si (KR)

(21) Appl. No.: 18/199,866

(22) Filed: May 19, 2023

(30) Foreign Application Priority Data
Nov. 1, 2022 (KR) 10-2022-0143953

(51) Int. Cl. A61L 9/20

(52) U.S. Cl. CPC A61L 9/20; A61L 2202/11; A61L 2202/122; A61L 2202/123; A61L 2202/14

(57) ABSTRACT

An air purifying performance evaluation device includes a chamber which accommodates an inspection object, a stirring fan which circulates air inside the chamber, a sterilizer configured sterilize microorganisms inside the chamber, a microorganism injector which injects microorganisms from the outside of the chamber into the inside of the chamber, and a sampling assembly which obtains a sample of microorganisms inside the chamber.

This application claims priority to Korean Patent Application No. 10-2022-0143953, filed on Nov. 1, 2022, and all the benefits accruing therefrom under 35 U.S.C. § 119, the content of which in its entirety is herein incorporated by reference.

### BACKGROUND

### 1. Field

The disclosure relates to an air purifying performance evaluation device.

### 2. Description of the Related Art

An air purifier is a device widely used in modern life to purify air by filtering physical particles such as dust, fine dust, and ultrafine dust, chemicals such as harmful gases, and microorganisms such as bacteria and viruses.

Air purifiers may be used for various purposes depending on their performance. As air purifiers are used more and more, the importance of their performance increases, and thus, it is desired to precisely evaluate the performance of the air purifiers.

### SUMMARY

Provided is an air purifying performance evaluation device.

Additional features will be set forth in part in the description which follows and, in part, will be apparent from the description, or may be learned by practice of the presented embodiments of the disclosure.

In an embodiment of the disclosure, an air purifying performance evaluation device includes a chamber which accommodates an inspection object, a stirring fan which circulates air inside the chamber, a sterilizer which sterilizes microorganisms inside the chamber, a microorganism injector which injects the microorganisms from outside of the chamber into the chamber, and a sampling assembly which obtains a sample of the microorganisms inside the chamber. The sampling assembly includes an extraction tube which extracts air containing microorganisms from inside the chamber to the outside of the chamber, and a sampler which collects microorganisms in the air extracted through the extraction tube from the outside of the chamber.

In an embodiment, the sampler may collect viruses outside the chamber.

In an embodiment, the sampler may include a filter which filters the viruses.

In an embodiment, the sampling assembly may include a sampling port for generating a flow of the air and a transfer tube for transferring the air from inside the chamber to the sampling port, the sampling port includes a first nozzle, a second nozzle, and a third nozzle, the first nozzle may be connected to the extraction tube to extract the air containing the microorganisms from inside the chamber to the outside of the chamber, the second nozzle may be connected to the transfer tube, the third nozzle may return the air inside the chamber transferred from the transfer tube to the sampling port to the inside of the chamber.

In an embodiment, the sampler may collect viruses outside the chamber, and one end of the extraction tube may be connected to the sampler.

In an embodiment, the sampling assembly may include a bypass tube including one end connected to the third nozzle of the sampling port and an opposite end disposed inside the chamber, and the sampling port may include a flow control device which adjusts a flow path inside the sampling port so that the air inside the chamber transferred from the transfer tube to the sampling port through the second nozzle is selectively discharged to the first nozzle or the third nozzle.

In an embodiment, the sampling assembly may include a transfer pipe, the transfer tube connected to the second nozzle may be connected to one end of the transfer pipe, and an opposite end of the transfer pipe may be disposed in a center of an inner space of the chamber.

In an embodiment, the sampling assembly may include a local sterilizer which sterilizes microorganisms inside the sampling port.

In an embodiment, the local sterilizer may be disposed opposite to the first nozzle of the sampling port.

In an embodiment, the local sterilizer may be a light-emitting diode (“LED”) lamp emitting light of a wavelength of about 200 nanometers (nm) or more and about 280 nm or less.

In an embodiment, a volume inside the chamber may be about 0.1 cubic meter (m³) or more and about 0.5 m³ or less.

In an embodiment, the sampler may include a sampling pump which creates an air flow through the extraction tube out of the chamber, and the sampling pump may generate a flow rate of about 1 liter per minute (L/min) or more and about 10 L/min or less and generate a negative pressure in the extraction tube to create an air flow from the first nozzle to the extraction tube.

In an embodiment, the sampling port may include a bypass pump that generates an air stream to return air inside the chamber delivered to the sampling port into the chamber.

In an embodiment, the sampling assembly may include a high efficiency particulate air (“NEPA”) filter which filters the microorganisms, and the HEPA may filter filters out the microorganisms contained in the air returned from the third nozzle to the inside of the chamber.

### BRIEF DESCRIPTION OF THE DRAWINGS

The above and other features and advantages of illustrative embodiments of the disclosure will be more apparent from the following description taken in conjunction with the accompanying drawings, in which:

FIG. 1 is a diagram for explaining an embodiment of an air purifying performance evaluation device;

FIG. 2 is a diagram for explaining an embodiment of a sampling assembly included in an air purifying performance evaluation device;

FIG. 3 is a view for explaining an embodiment of a microorganism injector included in an air purifying performance evaluation device;

FIG. 4 shows an embodiment of an air purifying performance evaluation device;

FIG. 5 is a diagram for explaining an embodiment of a sampling port included in an air purifying performance evaluation device;

FIG. 6 is a diagram for explaining an embodiment of a local sterilizer included in an air purifying performance evaluation device;

FIG. 7 is a diagram for explaining an embodiment of a local sterilizer included in an air purifying performance evaluation device.

FIG. 8 is a diagram for explaining an embodiment of a transfer pipe and a bypass tube included in an air purifying performance evaluation device;

FIG. 9 is a diagram for explaining an embodiment of an air purifying performance evaluation method; and

FIG. 10 is a view for explaining an embodiment of operations of a bypass flow path and a local sterilizer included in an air purifying performance evaluation device.

### DETAILED DESCRIPTION

Reference will now be made in detail to embodiments, illustrative embodiments of which are illustrated in the accompanying drawings, wherein like reference numerals refer to like elements throughout. In this regard, the illustrated embodiments may have different forms and should not be construed as being limited to the descriptions set forth herein. Accordingly, the embodiments are merely described below, by referring to the drawing figures, to explain features. As used herein, the term “and/or” includes any and all combinations of one or more of the associated listed items. Expressions such as “at least one of,” when preceding a list of elements, modify the entire list of elements and do not modify the individual elements of the list.

Hereinafter, an air purifying performance evaluation device according to various embodiments is described in detail with reference to the accompanying drawings. In the following drawings, the same reference numerals denote the same components, and the size of each component in the drawings may be exaggerated for clarity and convenience of description. Terms such as first and second may be used to describe various components, but the components should not be limited by the terms. Terms are only used to distinguish one component from another.

Singular expressions include plural expressions unless the context clearly dictates otherwise. In addition, when a certain component is said to “include”, this means that it may further include other components without excluding other components unless otherwise stated. In addition, the size or thickness of each component in the drawings may be exaggerated for clarity of explanation.

The use of the term “the” and similar denoting terms to refer to previously mentioned terms may be both singular and plural.

Operations including a method may be performed in any suitable order, unless expressly stated that they must be performed in the order described. In addition, the use of all exemplary terms (e.g., etc.) is simply for explaining technical ideas in detail, and the scope of rights is not limited due to these terms unless limited by claims.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). The term such as “about” can mean within one or more standard deviations, or within ±30%, 20%, 10%, 5% of the stated value, for example.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

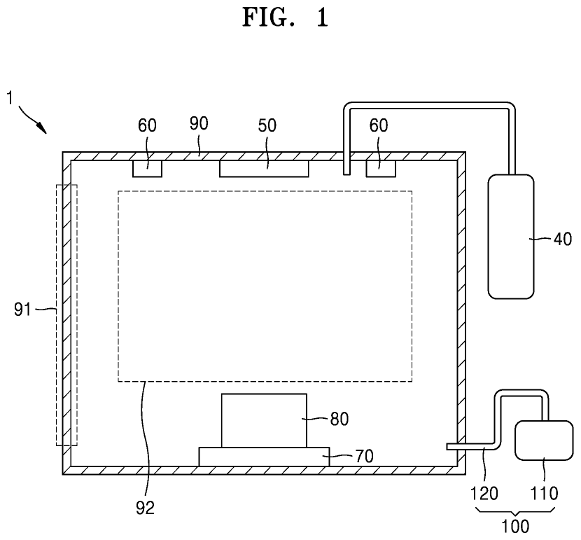

FIG. 1 is a diagram for explaining an embodiment of an air purifying performance evaluation device.

Referring to FIG. 1, the air purifying performance evaluation device 1 may include a chamber 90, a microorganism injector 40 and a sampling assembly 100.

The internal volume of the chamber 90 may be greater than or equal to about 0.1 cubic meter (m³) and less than or equal to about 1 m³. The internal volume of the chamber 90 may be greater than or equal to about 0.1 m³ and less than or equal to about 0.5 m³. The chamber 90 may have a quadrangular (e.g., rectangular parallelepiped shape). However, the shape of the chamber 90 is not limited thereto. The bottom of the chamber 90 may be a square with a width of 0.5 meter (m) and a length of 0.5 m, and the height of the chamber 90 may be 0.6 m. In this case, the internal volume of the chamber 90 may be 0.15 m³. The chamber 90 may be sealed. The pressure of the chamber 90 may be kept constant. A pressure regulating device (not shown) may be present at the upper portion of the chamber to maintain a constant pressure in the chamber 90. The pressure regulating device may include a high efficiency particulate air (“NEPA”) filter or a syringe filter.

At least one of the walls of the chamber 90 may include a glass wall 91 for internal observation. The glass wall 91 may have an ultra-violet (“UV”) blocking coating. A door 92 through which an inspection object 80 may be inserted or removed may be provided on a wall surface of the chamber 90.

The inspection object 80 may be an air purifier, an air sterilizer, an air purifying unit, an air sterilizing unit, a finished product, or a module. A method of purifying or sterilizing air by the inspection object 80 may be a plasma method or a ceramic catalytic filter method. The inspection object 80 may sterilize at least one of airborne microorganisms, e.g., bacteria and viruses. The inspection object 80 may be disposed on the inner bottom surface of the chamber 90. The inspection object 80 may be placed on a height adjusting device 70 inside the chamber 90. The height adjusting device 70 may adjust the height of the inspection object 80 placed inside the chamber 90.

The microorganism injector 40 may inject microorganisms into the chamber 90 from the outside of the chamber 90. The microorganism injector 40 may inject microorganisms in the form of aerosol into the chamber 90. Microorganisms may include at least one of bacteria and viruses, for example.

At least one sterilizer 60 capable of sterilizing microorganisms may be provided inside the chamber 90. The sterilizer 60 may be an ultraviolet-C (“UVC”) lamp. The sterilizer 60 may be disposed in the upper portion of the chamber 90. The sterilizer 60 may operate before and after evaluating the air purifying performance of the inspection object 80 to sterilize the remaining microorganisms in the chamber 90. The operation of the sterilizer 60 may be stopped while the air purifying performance of the inspection object (also referred to as a test object) 80 is being evaluated.

A stirring fan 50 for circulating air may be provided inside the chamber 90. The stirring fan 50 may be disposed in an upper portion of the chamber 90. The stirring fan 50 may uniformly agitate the microorganisms injected into the chamber 90 by the microorganism injector 40 into the chamber 90.

The sampling assembly 100 may include a sampler 110 and an extraction tube 120 provided outside the chamber 90 to obtain a sample of microorganisms inside the chamber 90 from outside the chamber. One end of the extraction tube 120 may be connected to the sampler 110, and the other end of the extraction tube 120 may be connected to the chamber 90 to extract air including microorganisms inside the chamber 90 to the outside of the chamber 90. A method for the sampling assembly 100 to extract air containing microorganisms inside the chamber 90 may be a method by a pressure difference, a method by capillarity, or a method by diffusion. However, the extraction tube 120 extracts the air containing microorganisms inside the chamber 90 is not limited to the above description. The sampler 110 may collect microorganisms in the air extracted through the extraction tube 120 in the outside of the chamber 90.

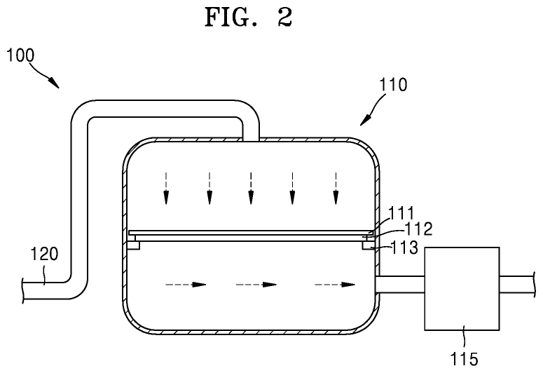

FIG. 2 is a diagram for explaining an embodiment of a sampling assembly included in an air purifying performance evaluation device.

Referring to FIG. 2, a sampling assembly 100 may move air from the extraction tube 120 toward the sampler 110. A sampling pump 115 may be provided on one side of the sampler 110. The sampling pump 115 may create an air stream out of the chamber 90 through the extraction tube 120. The sampling pump 115 may generate a flow rate of about 1 liter per minute (L/min) or more and about 10 L/min or less. The volume at which the sampling pump 115 samples the air inside the chamber 90 may be about 0.5% or more and about 2% or less of the chamber volume. The volume at which the sampling pump 115 samples the air inside the chamber 90 may be about 1% of the volume of the chamber. The sampling pump 115 may generate a negative pressure in the extraction tube 120 to create an air flow from the chamber 90 toward the extraction tube 120. Because the portion where the extraction tube 120 is connected to the sampler 110 is disposed on the upper surface of the sampler 110, the air supplied from the extraction tube 120 to the sampler 110 may be supplied from the upper portion to the lower portion of the sampler 110. The sampler 110 may collect viruses outside the chamber 90. The sampler 110 may include a hooking portion 113, a filter support unit 112 coupled to the hooking portion 113, and a filter 111 which is provided on the filter support unit 112 and filters viruses. The filter support unit 112 may support the filter 111 from a lower portion so that the filter 111 maintains its shape. The hooking portion 113 may fix the filter support unit 112 inside the sampler 110. The filter 111 may include a gelatin material. The filter 111 may include materials, such as polypropylene, polycarbonate, styrene, and polyvinylidene fluoride (“PVDF”).

The filter 111 may include a plurality of pores having a predetermined diameter. Each diameter of pores included in the filter 111 may be greater than or equal to about 0.2 micrometers (μm) and less than or equal to about 2 μm. The filter 111 may collect microorganisms through electrostatic attraction. Microorganisms collected in the filter 111 may be transferred to a nutrient medium together with the filter 111 and cultured. Viruses collected in the filter 111 may be transferred to a medium containing a host. The virus captured in the filter 111 may be diluted in a phosphate buffered saline (“PBS”) solution together with the filter 111. When the concentration of microorganisms collected in the filter 111 is diluted and analyzed, even a concentration of about 3×105 colony-forming unit per cubic centimeter (CFU/m³) or more may be measured. When the air purifying performance evaluation device may measure a microbial concentration of about 3×10 5 CFU/m³ or more, the initial microbial concentration of the chamber 90 may be set high. When the air purifying performance evaluation device may measure the microbial concentration of about 3×10 5 CFU/m³ or more, the air purifying rate of the inspection object 80 measurable by the air purifying performance evaluation device may be about 99.99% or more. In the operation of the air purifying performance evaluation device, when the initial microbial concentration is high, the effect of the natural reduction rate may be less.

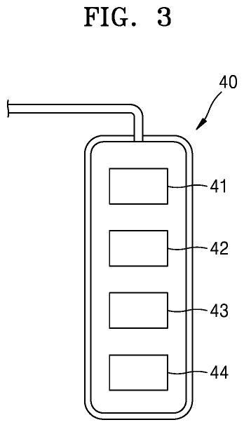

FIG. 3 is a diagram for explaining an embodiment of a microorganism injector.

Referring to FIG. 3, a microorganism injector 40 may include a dehumidifying device 41, an aerosol generator 42, an outdoor air pollution prevention filter 43, and a spray pump (also referred to as an injection pump) 44.

Referring to FIGS. 1 and 3, the injection pump 44 may suck outside air into the microorganism injector 40. The outdoor air pollution prevention filter 43 may remove contaminants such as microorganisms included in the outdoor air supplied to the microorganism injector 40. The microorganism injector 40 may inject air containing microorganisms into the chamber 90 by including microorganisms in the supplied external air. The injection pump 44 may inject air containing microorganisms into the chamber 90. The dehumidifying device 41 may prevent moisture from being included in microorganisms injected into the chamber 90. The aerosol generator 42 may inject microorganisms into the chamber 90 in the form of an aerosol. The aerosol generator 42 may include a nebulizer or atomizer.

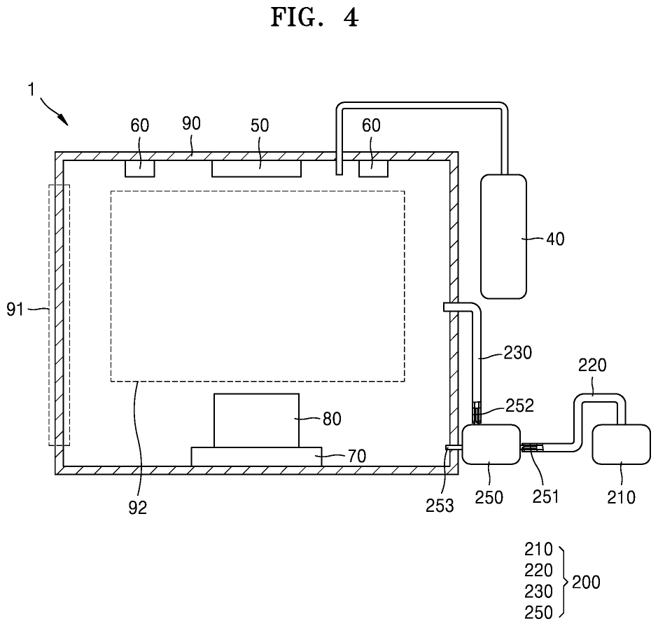

FIG. 4 shows an embodiment of an air purifying performance evaluation device.

Hereinafter, the description already given with reference to FIG. 1 is omitted and the description focuses on differences.

Referring to FIG. 4, a sampling assembly 200 may include a sampling port 250 and a transfer tube 230. The sampling port 250 may generate a flow of air. The transfer tube 230 may transfer air inside the chamber 90 to the sampling port 250. The transfer tube 230 and the sampling port 250 may be a single body.

The sampling port 250 may include a first nozzle 251, a second nozzle 252 and a third nozzle 253. The first nozzle 251 is connected to the extraction tube 220 to extract air containing microorganisms inside the chamber 90 to the outside of the chamber 90. The extraction tube 220 may extract air containing microorganisms inside the chamber 90 to the sampler 210. The second nozzle 252 may be connected to the transfer tube 230. The third nozzle 253 may return air inside the chamber 90 transferred from the transfer tube 230 to the sampling port 250 to the inside of the chamber 90. Sampling may be to extract air containing microorganisms in the chamber 90 into the sampler 210 through the transfer tube 230, the sampling port 250, and the extraction tube 220. Sampling may be performed multiple times. There may be microorganisms remaining in the transfer tube 230, the sampling port 250, and the extraction tube 220 after one unit of sampling is finished. In the case where a substantially high concentration of microorganisms remains in the transfer tube 230, the sampling port 250, and the extraction tube 220 before sampling, accuracy of sampling may deteriorate. The extraction tube 220 may be replaced with a new extraction tube 220 whenever one unit of sampling is finished. The sampling port 250 may include a bypass flow path from the second nozzle 252 toward the third nozzle 253 therein. The bypass flow path of the sampling port 250 may operate whenever sampling of one unit is completed. The air inside the chamber 90 sucked in by the transfer tube 230 may move through the bypass flow path and be returned to the inside of the chamber 90. When the bypass flow path operates, the concentration of microorganisms remaining in the bypass flow path inside the transfer tube 230 and the sampling port 250 may be similar to the concentration of microorganisms inside the chamber 90. The operation of the bypass flow path may last for about 1 minute. The flow rate of air circulated by the bypass flow path may be about 0.3 L/min or more and about 1 L/min or less. The flow rate of air circulated by the bypass flow path may be about 0.5 L. When the bypass flow path is operated after sampling of one unit is completed, the accuracy of sampling performed multiple times may be increased.

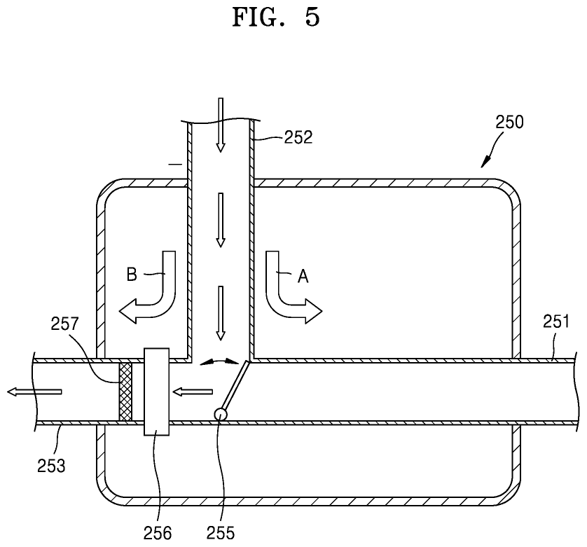

FIG. 5 is a diagram for explaining an embodiment of a sampling port.

Referring to FIG. 5, the sampling port 250 may include a flow control device 255, a bypass filter 256 and a bypass pump 257. The sampling port 250 may include a bypass flow path B directed from a second nozzle 252 to a third nozzle 253 and a sampling flow path A directed from the second nozzle 252 to a first nozzle 251. The air flow of the bypass flow path B and the sampling flow path A inside the sampling port 250 may be caused by one common pump (not shown). The flow control device 255 may control the flow path inside the sampling port 250 to selectively discharge the air inside the chamber 90 transferred from the transfer tube 230 to the sampling port 250 through the second nozzle 252 to the first nozzle 251 or the third nozzle 253. The bypass filter 256 may filter microorganisms in the air returned from the third nozzle 253 to the inside of the chamber 90 through the bypass flow path. The bypass filter 256 may be a HEPA filter. The bypass pump 257 may create an air flow in the bypass flow path. The bypass pump 257 may create an air flow to return the air inside the chamber 90 transferred to the sampling port 250 to the inside of the chamber 90. The bypass pump 257 may generate a flow rate of about 0.5 L/min or more about 4 L/min or less.

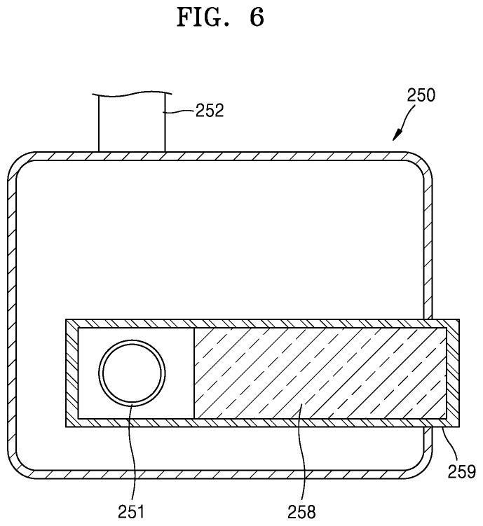

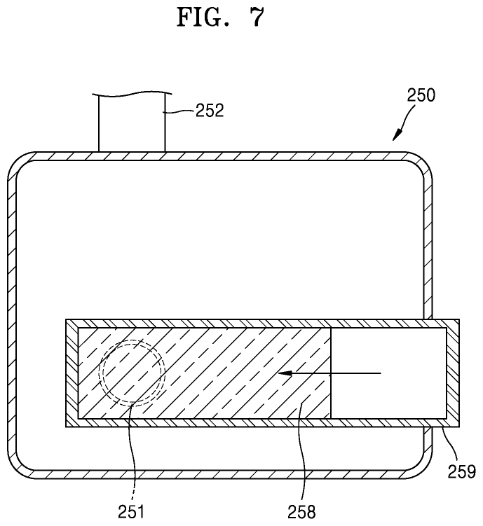

FIGS. 6 and 7 are views for explaining an embodiment of a local sterilizer included in an air purifying performance evaluation device.

Referring to FIGS. 5 to 7, a sampling port 250 may include a local sterilizer 258. The local sterilizer 258 may be disposed on a plane opposite to a first nozzle 251 of a sampling port 250. The local sterilizer 258 may sterilize microorganisms inside the sampling port 250. The local sterilizer 258 may sterilize the first nozzle 251. When the local sterilizer 258 sterilizes microorganisms inside the sampling port 250 after sampling of one unit is completed, the accuracy of sampling performed multiple times may be improved. The local sterilizer 258 may be a UVC light-emitting diode (“LED”) lamp. The local sterilizer 258 may be an LED lamp that emits light having a wavelength of about 200 nanometers (nm) or more and about 280 nm or less. The local sterilizer support unit 259 may support the local sterilizer 258. The local sterilizer support unit 259 may be a single body with the sampling port 250. The local sterilizer support unit 259 may be a single body with the chamber 90. The local sterilizer 258 may slide on the local sterilizer support unit 259. When the local sterilizer 258 is not operating, the local sterilizer 258 may move to the side of the first nozzle 251 on the local sterilizer support unit 259 so that the extraction tube 220 may be connected to the first nozzle 251.

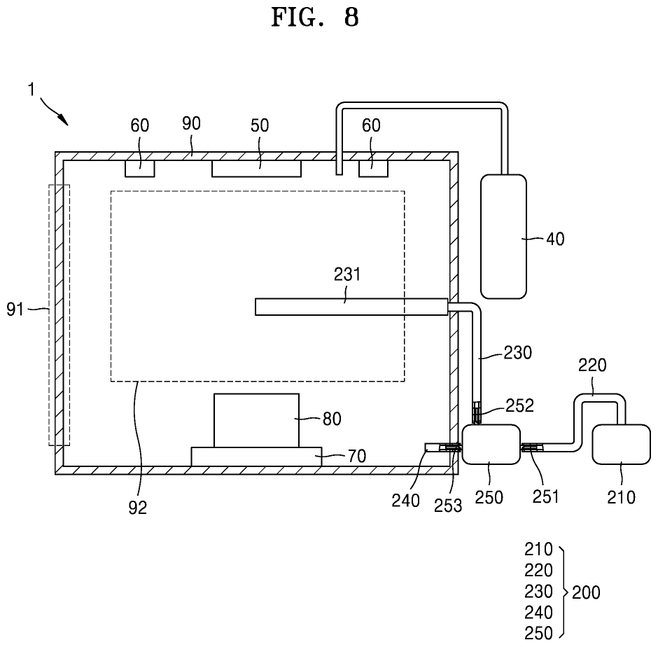

FIG. 8 is a view for explaining an embodiment of a transfer pipe and a bypass tube.

Referring to FIG. 8, a sampling assembly 200 may further include a transfer pipe 231 and a bypass tube 240. One end of the bypass tube 240 may be connected to the third nozzle 253 of the sampling port 250. The opposite end of bypass tube 240 may be disposed inside chamber 90.

A transfer tube 230 connected to a second nozzle 252 of the sampling port 250 may be connected to one end of the transfer pipe 231. The opposite end of the transfer pipe 231 may be disposed in the center of the inner space of the chamber 90. The transfer pipe 231 may suck air containing microorganisms inside the chamber 90. The transfer pipe 231 may include or consist of a metal material. When the transfer pipe 231 is disposed in the center of the inner space of the chamber 90, the transfer pipe 231 may well absorb air containing microorganisms inside the chamber 90.

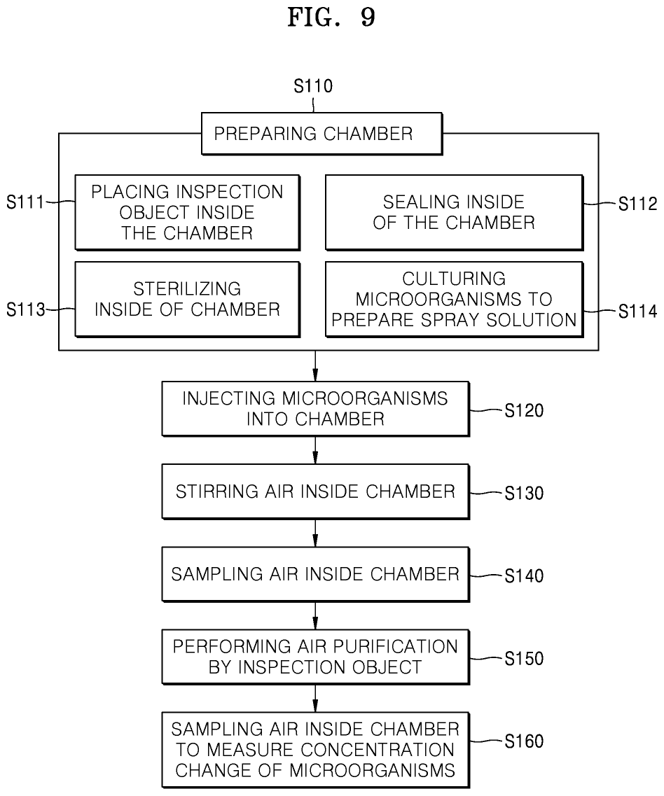

FIG. 9 is a diagram for explaining an embodiment of an air purifying performance evaluation method.

Referring to FIG. 9, the air purifying performance evaluation method in an embodiment may include preparing a chamber (S110), injecting microorganisms into the chamber (S120), stirring an air inside the chamber (S130), sampling the air inside the chamber (S140), performing air purification by an inspection object (S150), and sampling the air inside the chamber to measure a concentration change of microorganisms (S160).

Referring to FIGS. 1, 4, 8 and 9, preparing the chamber 90 (S110) may include placing the inspection object 80 inside the chamber 90 (S111), sealing the inside of the chamber 90 (S112), sterilizing the inside of the chamber 90 (S113), and culturing microorganisms to prepare a spray solution (S114). The operation of arranging the inspection object 80 inside the chamber 90 (S111) may be an operation of arranging an air purifier, an air sterilizer, an air purifying unit, or an air sterilizing unit in the center of the chamber 90. Placing the inspection object 80 inside the chamber 90 (S111) may include manipulating the height adjusting device 70. The operation of sealing the inside of the chamber 90 (S112) may be an operation of closing a door on the wall of the chamber 90. The operation of sterilizing the inside of the chamber 90 (S113) may include operating the sterilizer 60 inside the chamber 90.

Referring to FIGS. 3 and 9, the operation of culturing microorganisms to prepare a spray solution (S114) may be performed simultaneously with the operation of disposing the inspection object (also referred to as a specimen) 80 inside the chamber 90 (S111), the operation of sealing the inside of the chamber 90 (S112), and the operation of sterilizing the inside of the chamber 90 (S113), or may be performed separately. The operation of culturing the microorganisms to prepare the injection solution (S114) may include preparing the microorganism injector 40. The operation of culturing the microorganisms to prepare the spray liquid (S114) may include blowing clean air that has passed through the outdoor air pollution prevention filter 43 into the aerosol generator 42.

The operation of injecting microorganisms into the chamber 90 (S120) may be injecting microorganisms in an aerosol state into the chamber 90. The operation of injecting microorganisms into the chamber 90 (S120) may be injecting microorganisms into the chamber 90 from the top of the chamber 90.

Referring back to FIGS. 1, 4, 8 and 9, the operation of stirring the air inside the chamber 90 (S130) may include operating the stirring fan 50. The stirring fan 50 may operate for about 10 minutes. The operation of stirring fan 50 may include making the concentration of microorganisms inside the chamber 90 uniform. The operation of stirring the air inside the chamber 90 (S130) may further include a waiting operation. The waiting operation may be an operation of stabilizing the air inside the chamber 90 after the stirring fan 50 stops operating. The waiting operation may last for about 10 minutes to about 40 minutes. During the waiting operation, microorganisms may naturally be reduced.

The operation of sampling the air inside the chamber 90 (S140) may include extracting the air inside the chamber 90 at a rate of about 1 L or more and about 4 L or less per minute. The operation of sampling the air inside the chamber 90 (S140) may include extracting the air inside the chamber 90 to the sampler 110 using the extraction tube 120. The operation of sampling the air inside the chamber 90 (S140) may include extracting the air inside the chamber 90 transferred to the sampling port 250 through the transfer tube 230 to the sampler 210 through the extraction tube 220.

Referring to FIGS. 2 and 9, the operation of sampling the air inside the chamber 90 (S140) may include analyzing the concentration of microorganisms. The analyzing the concentration of microorganisms may include capturing the microorganisms. When the filter 111 collects bacteria, the operation of analyzing the concentration of microorganisms may include transferring the bacteria-collected filter 111 to a nutrient medium. When the filter 111 captures the virus, the operation of analyzing the concentration of microorganisms may include transferring the virus-collected filter 111 to a medium containing the host. The operation of analyzing the concentration of microorganisms may include transferring the virus-collected filter 111 to a PBS solution.

Referring back to FIGS. 1, 4, 8 and 9, the operation of performing air purification by the test object 80 (S150) may include operating the inspection object 80. The inspection object 80 may operate for a predetermined time. The inspection object 80 may lower the concentration of microorganisms inside the chamber 90.

The operation of sampling the air inside the chamber 90 to measure the change in concentration of microorganisms (S160) may include extracting the air from the inside the chamber 90 to be delivered to the sampler 110 through the extraction tube 120. The operation S160 may include extracting the air inside the chamber 90 transferred to the sampling port 250 through the transfer tube 230 into the sampler 210 through the extraction tube 220. The operation of sampling the air inside the chamber 90 to measure the change in concentration of microorganisms (S160) may include analyzing the concentration of microorganisms.

The operation of purifying the air by the inspection object 80 (S150) and the operation of sampling the air inside the chamber 90 to measure the concentration change of microorganisms (S160) may be repeated several times.

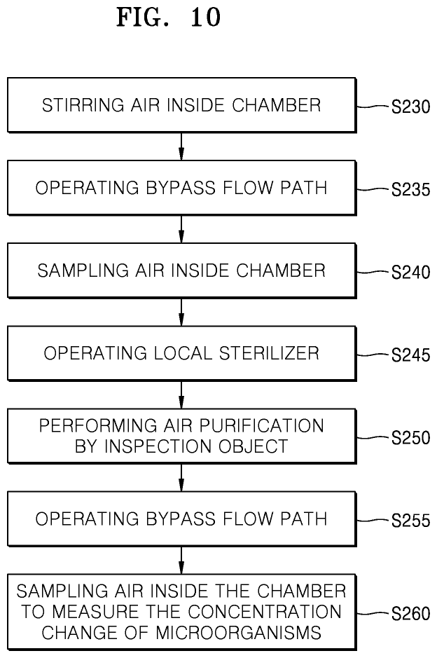

FIG. 10 is a diagram for explaining an embodiment of operations of a bypass flow path and a local sterilizer.

Referring to FIGS. 4, 8, and 10, before sampling the air inside the chamber 90 (S240), stirring an air inside the chamber (S230) and operating the bypass flow path (S235) may be included. The operating of the bypass passage (S235) may proceed for about 1 minute. Operating the bypass passage (S235) may include returning air inside the chamber 90 transferred through the transfer tube 230 to the sampling port 250 to the inside of the chamber 90 through the third nozzle 253. When the bypass flow path is operated, the concentration of microorganisms in the transfer tube 230 and the sampling port 250 may be the same as the concentration of microorganisms in the air inside the chamber 90.

An operation of performing air purification by an inspection object (S250) and operating the bypass flow path (S255) may be included before the sampling of the air inside the chamber 90 to measure the change in concentration of microorganisms (S260).

Referring to FIGS. 4, 6, 7, 8, and 10, after the sampling of the air inside the chamber 90 (S240), operating the local sterilizer 60 (S245) may be included. The operating of the local sterilizer 60 (S245) may include removing the extraction tube 220 from the first nozzle 251. The operating of the local sterilizer 60 (S245) may include disposing the local sterilizer 258 to face the first nozzle 251.

The operating of the local sterilizer 60 (S245) to the sampling of the air inside the chamber 90 to measure the concentration change of microorganisms (S260) may be repeated several times.

As described above, the air purifying performance evaluation device and method in the embodiment obtain air inside the chamber from the outside of the chamber, so that the performance of the air purifier may be easily evaluated.

It should be understood that embodiments described herein should be considered in a descriptive sense only and not for purposes of limitation. Descriptions of features or advantages within each embodiment should typically be considered as available for other similar features or advantages in other embodiments. While embodiments have been described with reference to the drawing figures, it will be understood by those of ordinary skill in the art that various changes in form and details may be made therein without departing from the spirit and scope as defined by the following claims.

An air purifying performance evaluation device in an embodiment may easily evaluate the performance of an air purifier, an air sterilizer, an air purifying module, an air sterilizing module, an air purifying unit, and an air sterilizing unit.

What is claimed is:

1. An air purifying performance evaluation device which evaluates air purifying performance of an inspection object by air and microorganisms, the air purifying performance evaluation device comprising:
a chamber which accommodates the inspection object;
a stirring fan which circulates the air inside the chamber;
a sterilizer which sterilizes the microorganisms inside the chamber;
a microorganism injector which injects the microorganisms from outside of the chamber into the chamber; and
a sampling assembly which obtains a sample of the microorganisms inside the chamber,
wherein the sampling assembly includes an extraction tube which extracts air containing the microorganisms from inside the chamber to the outside of the chamber, and a sampler which collects the microorganisms in the air extracted through the extraction tube from the outside of the chamber.

2. The air purifying performance evaluation device of claim 1, wherein the sampler collects viruses outside the chamber.

3. The air purifying performance evaluation device of claim 2, wherein the sampler includes a filter which filters the viruses.

4. The air purifying performance evaluation device of claim 1,
wherein the sampling assembly includes a sampling port for generating a flow of the air and a transfer tube for transferring the air from inside the chamber to the sampling port,
the sampling port includes a first nozzle, a second nozzle, and a third nozzle,
the first nozzle is connected to the extraction tube to extract the air containing the microorganisms from inside the chamber to the outside of the chamber,
the second nozzle is connected to the transfer tube, and
the third nozzle returns the air inside the chamber transferred from the transfer tube to the sampling port to the inside of the chamber.

5. The air purifying performance evaluation device of claim 4, wherein the sampler collects viruses outside the chamber, and one end of the extraction tube is connected to the sampler.

6. The air purifying performance evaluation device of claim 4,
wherein the sampling assembly includes a bypass tube including one end connected to the third nozzle of the sampling port and an opposite end disposed inside the chamber, and
the sampling port includes a flow control device which adjusts a flow path inside the sampling port so that the air inside the chamber transferred from the transfer tube to the sampling port through the second nozzle is selectively discharged to the first nozzle or the third nozzle.

7. The air purifying performance evaluation device of claim 4,
wherein the sampling assembly includes a transfer pipe,
the transfer tube connected to the second nozzle is connected to one end of the transfer pipe, and
an opposite end of the transfer pipe is disposed in a center of an inner space of the chamber.

8. The air purifying performance evaluation device of claim 4, wherein the sampling assembly includes a local sterilizer which sterilizes the microorganisms inside the sampling port.

9. The air purifying performance evaluation device of claim 8, wherein the local sterilizer is disposed opposite to the first nozzle of the sampling port.

10. The air purifying performance evaluation device of claim 8, wherein the local sterilizer is an LED lamp emitting light of a wavelength of about 200 nanometers or more and about 280 nanometers or less.

11. The air purifying performance evaluation device of claim 1, wherein a volume inside the chamber is about 0.1 cubic meter or more and about 0.5 cubic meter or less.

12. The air purifying performance evaluation device of claim 4,
wherein the sampler includes a sampling pump which creates an air flow through the extraction tube out of the chamber, and
the sampling pump generates a flow rate of about 1 liter per minute or more and about 10 liters per minute or less, and
generate a negative pressure in the extraction tube to create the air flow from the first nozzle to the extraction tube.

13. The air purifying performance evaluation device of claim 12, wherein the sampling port includes a bypass pump that generates an air stream to return air inside the chamber delivered to the sampling port into the chamber.

14. The air purifying performance evaluation device of claim 4,
wherein the sampling assembly includes a high efficiency particulate air filter for filtering the microorganisms, and
the high efficiency particulate air filter filters out the microorganisms contained in the air returned from the third nozzle to the inside of the chamber.

15. A method for evaluating an air purifying performance of an inspection object by air and microorganisms, the method comprising:
preparing a chamber;
injecting the microorganisms into the chamber;
stirring the air inside the chamber;
sampling the air inside the chamber,
performing air purification by the inspection object, and
sampling the air inside the chamber to measure a concentration change of the microorganisms.

16. The method according to claim 15, further comprising:
placing the inspection object inside the chamber.

17. The method according to claim 16, further comprising:
sealing the inside of the chamber.

18. The method according to claim 17, further comprising:
sterilizing the inside of the chamber.

19. The method according to claim 17, further comprising:
culturing the microorganisms to prepare a spray solution.

20. The method according to claim 16, wherein
placing the inspection object inside the chamber includes arranging an air purifier, an air sterilizer, an air purifying unit, or an air sterilizing unit in a center of the chamber.
